(19) United States
(12) Patent Application Publication
(10) Pub. No.: US 2015/0025119 A1
(43) Pub. Date: Jan. 22, 2015

(54) Release Application for Antihypertensive Agent on Plant Fiber Bundle

(71) Applicant: National Tsing Hua University, Hsin Chu City (TW)

(72) Inventors: Chen-Meng Kuan, Hsinchu County (TW); Chao-Min Cheng, New Taipei City (TW); Robert S. Langer, Cambridge, MA (US)

(73) Assignee: National Tsing Hua University, Hsin Chu City (TW)

(21) Appl. No.: 13/970,020

(22) Filed: Aug. 19, 2013

(30) Foreign Application Priority Data
Jul. 16, 2013 (TW) 102125458

(51) Int. Cl. A61K 9/70; A61K 31/41

(52) U.S. Cl. CPC A61K 9/70; A61K 31/41
USPC 514/381

(57) ABSTRACT

A method of releasing an antihypertensive agent on a plant fiber bundle is proposed. Firstly, the antihypertensive agent is fixed onto the plant fiber bundle. Then, the antihypertensive agent is fixed onto the plant fiber bundle and released in a buffer solution system. Next, fluorescence analysis of released antihypertensive agent is performed to evaluate the release efficiency of the antihypertensive agent.

### TECHNICAL FIELD

The present invention is generally relevant to an agent release method, specifically, a method of releasing an antihypertensive agent from a plant fiber bundle base.

### BACKGROUND

Hypertension is the main risk factor for cardiovascular diseases. Patients with cardiac diseases or apoplexy often have hypertension symptoms before the onset of their illnesses. Hypertension is also the main cause of cardiac failure in the elderly population. Therefore, people aged over 60 years generally need antihypertensive drugs to suppress or control high blood pressure caused by hypertension in daily life.

In 2000, about 26.4 percent of adults worldwide had hypertension symptoms, and the percentage is expected to rise to 29.2% by AD 2025 (Kearney et al., 2005, The Lancet, 365: 217-223). Hypertension symptoms are considered an important risk factor for cardiovascular diseases such as myocardial infarction, apoplexy, heart failure, terminal diabetes, and other diseases. Each 5 mmHg reduction in blood pressure reduces the probability of cardiovascular diseases by 16% (FitzGerald et al., 2004, J. Nutr. 134: 980S-988S). The report of the seventh Joint National Committee, JNC7 indicates that a systolic blood pressure/diastolic blood pressure higher than 115/75 mmHg increases the probability of heart disease and apoplexy. The Committee suggested that people with high blood pressure (systolic blood pressure 120-139 mmHg or diastolic blood pressure of 80-89 mmHg) should adopt a healthy lifestyle to avoid cardiovascular diseases.

Some hypertension patients also need antihypertensive drugs or a change it diet to suppress blood pressure in everyday life. Generally, antihypertensive drugs are often used before or after dinner. For busy modern people, however, using antihypertensive medication capsules or drugs is very inconvenient. Failure to take the drugs on time can cause abnormally elevated blood pressure and adverse health impacts.

To address these shortcomings, a new method is proposed for releasing anti-hypertensive agents.

### SUMMARY OF THE INVENTION

To address the above shortcomings, a method is proposed for releasing anti-hypertensive agents through a platform (base) of plant fiber bundles.

Another objective of the invention is to provide a release application for antihypertensive agent on a plant fiber bundle to facilitate patients in taking the correct antihypertensive agent dose and to facilitate manufacturing of the agent.

One feature of the invention is its potential use for releasing antihypertensive agents from a base of plant fiber bundles. After the antihypertensive agents fixed onto a base of plant fiber bundle were released in a buffer solution, a fluorescence analysis of the anti-hypertensive agents was performed.

Another potential application is in determining the relation (e.g., linear relation) between the antihypertensive agent concentration and the mean fluorescence intensity of the antihypertensive agents.

The process used in the proposed fixing step includes coating or impregnating the plant fiber bundle base with a liquid antihypertensive agent liquid.

The buffer solution comprises physiological saline. The materials of the plant fiber bundle base comprise natural fibers, wood fibers, plant fiber composites, or composite nano-fibers. The plant fiber bundle base comprises a toothpick. After release of the anti-hypertensive agents, fluorescence analysis is performed under the conditions of excitation wavelength, 259 nanometers and emission wavelength, 385 (or 399) nanometers.

### BRIEF DESCRIPTION OF THE DRAWINGS

The attached specifications and drawings outline the preferred embodiments of the invention, including the details of its components, characteristics and advantages.

FIG. 1 shows a process flow for releasing antihypertensive agents onto a plant fiber bundle base according to one embodiment of the invention;

FIG. 2a shows the results of a statistical analysis of mean fluorescence intensity of various Valsartan concentrations;

FIGS. 2b-c are fluorescence images of Valsartan concentrations of 20 and 2.5 mg/mL, respectively;

FIG. 3 illustrates the relation between Valsartan concentration and mean fluorescence intensity of Valsartan;

FIG. 4 illustrates the proportion of release (release percentage) of Valsartan over time.

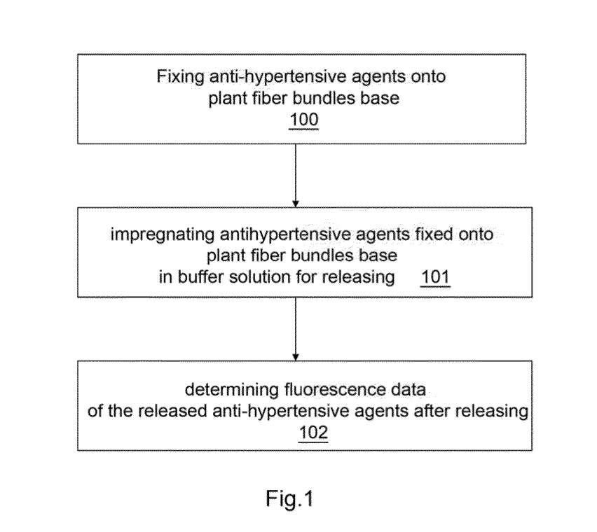

### DETAILED DESCRIPTION

Next, the preferred embodiments of the invention are described in further detail. Notably, however, the preferred embodiments are provided for illustration purposes rather than for limiting the use of the invention. The invention is also applicable in many other embodiments besides those explicitly described, and the scope of the invention is not expressly limited except as specified in the accompanying claims.

The invention provides an inexpensive but robust and easily performed approach for fabricating an antihypertensive agent on a plant fiber bundle base (platform) or for releasing an antihypertensive agent from a plant fiber bundle base. For example, the antihypertensive agent Valsartan can be released from a plant fiber bundle base such as a toothpick. Other antihypertensive agents include, but are not limited to, Eprosartact (Epro), Azilsartan, Candesartan, (Normodyne, Trandate), L-type calcium channel, Remikiren, Telmisartan and Reserpine.

Plant fiber is intrinsic sclerenchyma of seed plant widely distributed therein. Generally, plant fibers are characterized by slender cells that are sharp at both ends and with a thick wall and a secondary wall with a simple pit. Plant fiber mainly provides mechanical support for a plant, and plant fiber bundle is used to absorb moisture. Accordingly, the invention uses plant fiber bundles as an adsorption base to take advantage of the capability of plant fiber bundles to adsorb liquid antihypertensive agents.

The invention enhances the physical adsorption properties of plant fiber bundles to facilitate fixing of antihypertensive agents (Valsartan) thereto, and fluorescence of antihypertensive agents (Valsartan) can be observed under the conditions of wavelength (λ) 259 nm (nanometer), 385 nm, or 259 nm, 399 nm. According to this feature, an optical instrument is used to collect fluorescence data for varying concentrations of Valsartan attached to toothpicks. The analysis of fluorescence intensity was performed using ImageJ image analysis software, and a calibration curve is further performed. The proposed method for releasing an antihypertensive agent from a plant fiber bundle base is described further below.

Firstly, in step 100, antihypertensive agents are fixed onto a plant fiber bundle base such as a toothpick, i.e., solid antihypertensive agents attached onto the plant fiber bundle base. In this step, the plant fiber bundle base may be impregnated in liquid antihypertensive agents. Because of the physical adsorption properties of plant fiber bundles, the composition of the liquid antihypertensive agents is gradually adsorbed into the plant fiber bundles until the amount of antihypertensive agents on plant fiber bundle base reaches a saturated condition. In one embodiment, the concentration of antihypertensive agents (e.g., Valsartan) in liquid form is 2.5, 5, 8, 10, 20 mg/mL, respectively. Then, the plant fiber bundle base is removed. The plant fiber bundle base is naturally dried, or cured by light or heat energy, to finish (form) the plant fiber bundle device with antihypertensive agents attached thereto.

In another embodiment, antihypertensive agents may be attached onto the plant fiber bundle base by a coating process.

The plant fiber bundle base must have high capacity to adsorb antihypertensive agents. For example, the materials of the plant fiber bundle base may include, but are not limited to, natural fibers, wood fibers, plant fiber composites, composite nano-fibers. One example of a wood fiber material is a toothpick. Raw materials of plant fiber of plant fiber composite may be selected from the following components: sugarcane residue, maize core, coconut shell, peanut shell, leaves of tree or bamboo, dust of timber or bamboo or a combination thereof; the above-mentioned raw materials of plant fiber can be used to form a plant fiber composite. Composite nanofiber is for example, carbon nanofiber, which is hollow and has a diameter of 5-20 nanometer (nm). Because of its high surface area and high porosity, carbon nanofiber is an excellent adsorbent.

For example, raw materials of the plant fiber component may be washed to remove dust and mud and then dried, chopped and pulverized. The short fiber component may be composed of rice husk, bran of wheat or sorghum, peanut shell and leaves of tree or bamboo, or dust of timber or bamboo, which is directly subjected to pulverize to obtain plant fiber raw material. The plant fiber raw material, starch auxiliary and biological polymer additive are mixed well, kneaded and compressed at a low temperature in a pressurized condition, extruded to form granules, and cooled and sieved by a sieve with an appropriate pore size to obtain the plant fiber composite.

Another example of wood fiber material is Arundo donax, a high and erect perennial herb. Arundo donax is formed into a wood fiber material after drying. Stems of Arundo donax are hollow and have wall thicknesses of 2-7 millimeter. Sections are divided by nodes. The length of each section of Arundo donax is 12 to 30 centimeters. Fiber bundles of Arundo donax are freely distributed throughout a cross-sectional area of its base parenchyma cells. The number of fiber bundles around the stem of Arundo donax is larger than that facing towards inside of Arundo donax. The size of fiber bundles around the stem of Arundo donax is smaller than that of facing towards inside of Arundo donax.

Subsequently, in step 101, antihypertensive agents fixed onto the plant fiber bundle base are impregnated in buffer solution for releasing. The buffer solution or dissolving solution may be physiological saline. In this step, the plant fiber bundle base is immersed in physiological saline. In one embodiment, antihypertensive agents fixed onto the plant fiber bundle base are formed by the concentration of 2.5, 5, 8, 10, 20 mg/mL, respectively, of antihypertensive agents (e.g., Valsartan) in liquid form Therefore, antihypertensive agents are released in physiological saline.

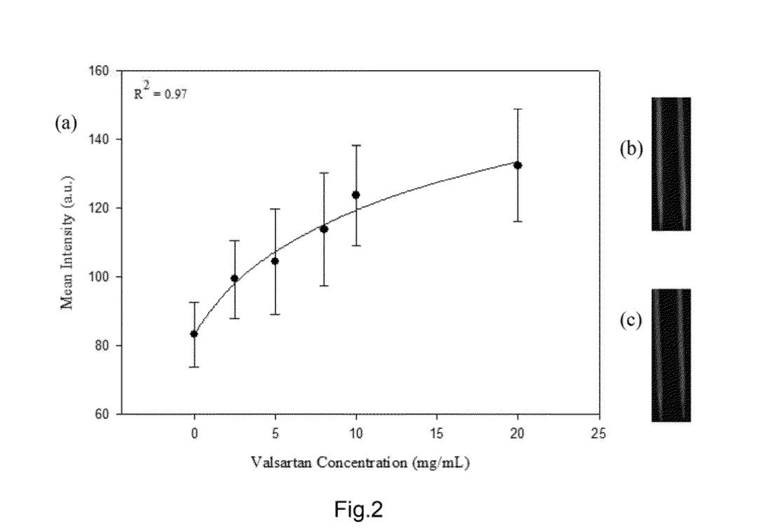

Next, in step 102, fluorescence analysis of the released anti-hypertensive agents is performed. As described in the literature (Journal of Pharmaceutical and Biomedical Analysis, 26 (2001) 477-486; pKa determination of angiotensin II receptor antagonists (ARA II) by spectrofluorimetry; Biomed. Chromatogr 2011; 25: 1252-1259; High-performance liquid chromatographic determination of antihypertensive drugs on dried blood spots using a fluorescence detector-method development and validation), the fluorescence of the anti-hypertensive agent (Valsartan) was observed at an excitation wavelength (λ) of 259 nm and an emission wavelength of 385 nm or at an excitation wavelength of 259 nm and an emission wavelength of 399 nm. Based on this feature, an optical instrument is used for fluorescence analysis of different concentrations of Valsartan affixed to toothpicks. Fluorescence analysis of different concentrations of Valsartan was performed using ImageJ image analysis software. FIG. 2a shows the mean intensity results of a statistical analysis (n=64, number of measurement) of Valsartan concentrations 2.5, 5, 8, 10, 20 mg/mL and its calibration curve.

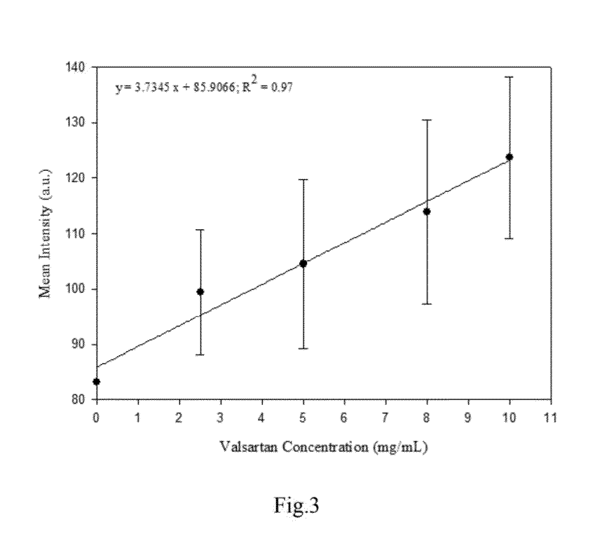

FIGS. 2b-c show fluorescence images of Valsartan at concentrations of 20 and 2.5 mg/mL, respectively. The results of calibration curve of FIG. 2a shows that the fluorescence intensity of Valsartan approaches saturation at concentrations from 10 to 20 mg/mL. The following linear equation was established at Valsartan concentrations of 0, 2.5, 8, 10 mg/mL (FIG. 3):

y=3.7345 x+85.9066

where y is Valsartan concentration and x is mean intensity of fluorescence. The linear equation indicates the relation between Valsartan concentration and mean fluorescence intensity of Valsartan.

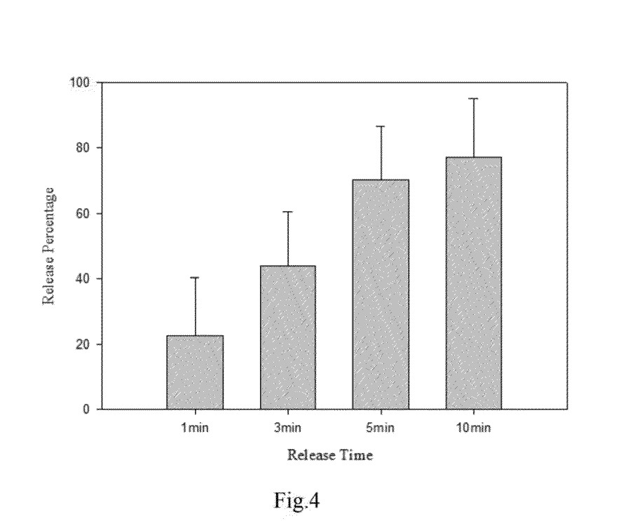

In one embodiment, the antihypertensive agent (here, Valsartan) is impregnated at a concentration of 10 mg/mL in physiological saline for release after 1, 3, 5, 10 minutes. An optical instrument is used to collect fluorescence data of Valsartan after release in physiological saline in 38 repetitions of the experiment. FIG. 4 shows that, as time increases, the proportion of release (release percentage) of Valsartan also increases until the saturation level is reached, but the released amount is not increased. According to the established linear equation, the efficiency of antihypertensive agent release can be estimated; additionally, by controlling the release time, the buffer solution concentration, etc., the efficiency of antihypertensive agent release and the released amount of antihypertensive agent can be precisely controlled.

As noted above, the present invention provides an inexpensive but robust and easily performed approach for various applications. The advantages and features of the proposed method include the following:
1. The use of different plant fiber bundle types may have different capacity to adsorb antihypertensive agents and different saturation levels;
2. Using different buffer solutions for antihypertensive agents may differ in their release of antihypertensive agents and saturation level;
3. The relation (such as linear relation) between Valsartan concentration and mean fluorescence intensity of Valsartan may be determined;
4. According to the established linear equation, the efficiency of antihypertensive agent release can be estimated; by controlling the release time, the buffer solution concentration, the efficiency in antihypertensive agent release and the released amount of antihypertensive agent can be controlled.

For a person skilled in the art, the preferred embodiments described above are illustrations rather than limitations of the applications of the invention. The invention is intended to enable various modifications, and similar arrangements are included within the spirit and scope of the appended claims, the scope of which should be accorded the broadest interpretation so as to encompass all such modifications and similar structures.

What is claimed is:

1. A method for releasing antihypertensive agents onto plant fiber bundles base, comprising:
fixing antihypertensive agents onto a plant fiber bundles base; releasing said antihypertensive agents fixed onto plant fiber bundles base in a buffer solution; and determining fluorescence data of said antihypertensive agents after releasing.

2. The method of claim 1, further comprising a step of creating a relation between concentration of said antihypertensive agents and mean intensity of fluorescence of said antihypertensive agents.

3. The method of claim 2, wherein said relation is a linear relation.

4. The method of claim 1, wherein said step of fixing includes impregnating said plant fiber bundles base in antihypertensive agents liquid.

5. The method of claim 1, wherein said step of fixing includes a coating process.

6. The method of claim 1, wherein buffer solution comprises physiological saline.

7. The method of claim 1, wherein materials of said plant fiber bundles base comprise natural fibers, wood fibers, plant fiber composites, or composite nano-fibers.

8. The method of claim 1, wherein said plant fiber bundles base comprises toothpick.

9. The method of claim 1, wherein said determining fluorescence data of said antihypertensive agents after releasing is performed under the conditions of excitation wavelength 259 nanometer and emission wavelength 385 nanometer.

10. The method of claim 1, wherein said determining fluorescence data of said antihypertensive agents after releasing is performed under the conditions of excitation wavelength 259 nanometer and emission wavelength 399 nanometer.
